(19) United States
(12) Patent Application Publication
(10) Pub. No.: US 2013/0035682 A1
(43) Pub. Date: Feb. 7, 2013

(54) Noninvasive Nerve Ablation

(71) Applicant: Michael D. Weil, Half Moon Bay, CA (US)

(72) Inventor: Michael D. Weil, Half Moon Bay, CA (US)

(73) Assignee: Sirius Medicine, LLC, Half Moon Bay, CA (US)

(21) Appl. No.: 13/565,788

(22) Filed: Aug. 2, 2012

Related U.S. Application Data
(60) Provisional application No. 61/514,207, filed on Aug. 2, 2011.

(51) Int. Cl. A61B 18/18

(52) U.S. Cl. 606/33

(57) ABSTRACT

The present invention relates to the field of medical imaging and therapy of nerve lesions that are detrimental to the body. The method incorporates both identifying imaging for localization and treatment with a radiation source. Clear images of individual nerve structures are fused to conventional images for accurate treatment planning with noninvasive ablating beams. This results in increased radiation dose in the lesion. Furthermore, neither endovascular intervention nor additional internally introduced reference points are needed. More specifically this invention relates to a method for optimizing delivery of ablating beams to a lesion compared to the surrounding normal tissues without invasive techniques.

### RELATED U.S. APPLICATION DATA

This application claims priority of provisional application No. 61/514,207 filed Aug. 2, 2011 and entitled, Noninvasive Nerve Ablation.

### REFERENCES CITED

U.S. PATENT DOCUMENTS

| | | |
|---|---|---|
| 5,560,360 | October 1996 | Filler et al. |
| 5,706,813 | January 1998 | Filler et al. |
| SN 13/009,784 | January 2011 | Beetel et al. |
| 8,175,711 | May 2012 | Demarais et al. |

### OTHER PUBLICATIONS

Smithwick, “Surgical treatment of hypertension,” Am. J. Med. 4:744, 1948, Elsevier.

Chobanian, “The hypertension paradox—more uncontrolled disease despite improved therapy,” NEJM 361:878, 2009, Mass. Med. Soc.

### FIELD OF INVENTION

This invention relates to the field of medical therapy to localize and noninvasively ablate nerves with ionizing radiation to ameliorate pathophysiologic processes.

BACKGROUND

Sympathetic Trunk—The involuntary, or autonomic, nervous system comprises the fight-or-flee responses of the sympathetic and parasympathetic divisions, respectively. The sympathetic nervous system runs as bilateral paraspinal trunks from the stellate ganglia in the neck down to the lumbar region. FIG. 1 depicts the anatomical relationship of the sympathetic trunk 10 is as follows: The spinal cord runs through the center of the foramen formed by the bony vertebral bodies 5. Coming approximately horizontally off the spinal cord when a person is upright are the dorsal and ventral roots, which merge to form the spinal nerves 7. In addition, there are two vertically running nerve chains parallel and on either side of the spinal cord. These vertically directed paraspinal nerves are the sympathetic trunk 10. The trunk bulges approximately midway between each intersection with the horizontal spinal nerves 7 to form the sympathetic ganglia. (Ganglia are relay points composed of nerve cell bodies and nerve processes connected together and can collect together to form a plexus.)These particular ganglia are identified as the paravertebral ganglia 12. The sympathetic trunk 10 crosses the spinal nerves 7, but the nerves do not run through one another. There are two small nerves, the more lateral white (myelinated) rami communicans 8 and the more medial gray rami communicans 9, which run between each spinal nerve 7 and the sympathetic ganglia. At the level of each ventral root, the rami communicans carry the axons of the pre-ganglionic and the post ganglionic neurons, to and from the sympathetic ganglia, respectively. In general, the post ganglionic neurons from the paravertebral ganglia 12 innervate the skin blood vessels and glands. In addition, there are preganglionic neurons, which travel through the paravertebral ganglia 12 and then via the splanchnic nerves 18, e.g., the lesser and least splanchnic nerves 18, to contact post ganglionic neurons in ganglia that are separate from the sympathetic truck. The separate sympathetic ganglia are known as collateral ganglia or the prevertebral ganglia 22, and innervate organs such as the heart, kidney 30 and gastrointestinal tract. These include the celiac ganglia, superior mesenteric ganglion, aorticorenal ganglion, renal ganglion and inferior mesenteric ganglion. The sympathetic trunk 10 and the paravertebral ganglia 12 are not accessible by intravascular or endovascular techniques. Likewise, many of the prevertebral ganglia 22 and plexuses are also not accessible by intravascular or endovascular methods.

Surgical literature dating back nearly a century documents the efficacy of ablating sections of the sympathetic trunk 10 to reduce high blood pressure. This is apparently the result of lowering levels of norepinephrine flowing into the kidneys 30 as well as a reduction in vascular tone. Surgical sympathectomy for the treatment of hypertension was practiced for several decades prior to the development of diuretics in the 1950s (Smithwick, “Surgical treatment of hypertension,” Am. J. Med. 4:744, 1948, Elsevier; Chobanian, “The hypertension paradox—more uncontrolled disease despite improved therapy,” NEJM 361:878, 2009, Mass. Med. Soc.). It was known at the time that three factors affected peripheral resistance in hypertensive patients including nervous, humoral and vascular disease. The minimal surgical procedure recommended then was bilateral removal of the sympathetic trunks 10 from T8-L1, and was known as thoracolumbar splanchnicectomy. The great splanchnic nerves 18 were removed from the mid-thoracic level to the celiac axis, but sometimes modified to bilateral removal of the sympathetic trunks 10 from the inferior cervical to the twelfth thoracic ganglia inclusive. This resulted in a lowering of the diastolic pressure and narrowing of the pulse pressure. The denervated areas did not perspire while sweating was increased in the intact areas. Postural hypotension with the rapid heart rate along with cold hands could result. These changes apparently disappeared after 4-6 months. Some patients had persistent tachycardia after exercise. Surgical intervention on the sympathetic nervous system appeared to slow the progress of hypertension.

Surgical sympathectomy is presently performed for excessive sweating of the hands and feet. Endoscopic techniques are used to place surgical clips above and below the sympathetic trunk 10 at T3-T4 and L3-L4 levels, respectively. Facial sweating is treated with a T2 sympathectomy just below the stellate ganglion. A T2 and T3 sympathectomy will effectively treat palmar hyperhidrosis. Treatment of axial sweating uses a T3, T4 and sometimes T5 sympathectomy. Endoscopic thoracic sympathectomy has been used to treat refractory cardiac tachyarrhythmias. Such procedures carry some risk as the thoracic sympathetic trunk 10 crosses over the intercostal vessels care must be taken not to lacerate them, and traction on the stellate ganglion can produce a Homer's syndrome. The lumbar approach is made below the tip of the 11^{th} rib. On the other hand, spinal stimulation has largely replaced sympathectomy for the treatment of pain syndromes and vasculitis.

Recently nonsurgical, but invasive techniques have demonstrated efficacy in attenuating sympathetic output and as a result lower blood pressure in medically unresponsive hypertension. Catheters with radiofrequency probes at their tip have been inserted into the renal artery via the aorta (U.S. Pat. No. 8,175,711). The angulated probe was then rotated and slowly withdrawn from the artery while radiofrequency pulses were delivered through the artery to heat the nerves in the outer part of the vessel wall. The branches of the renal nerve were heated to destructive temperatures of 60° C. The lining of the artery as well as the arterial wall were spared high temperatures apparently due to blood flow within the artery dissipating the heat. Although renal artery catheterization is significantly less invasive then surgical sympathectomy, it does have a risk of blood clots, bleeding and damage to the blood vessels. In addition, the renal nerves themselves are not visualized and the actual temperature change via heating delivered to the outer wall of the renal artery is not definitely known. For instance, the power used can produce different heating effects depending on varying arterial blood flow.

In addition to radiofrequency ablation for hypertension treatment, destruction of the perirenal nerves has been performed with a specialized catheter and needle, which penetrates the vessel wall and injects guanethidine into the adventitia to destroy the nerve. Cryoablation, as well as high-intensity focused ultrasound (HiFU) have also been proposed as methods to ablate the sympathetic renal nerves. In the case of HiFU, it has been suggested that the perirenal sympathetic nerves can be destroyed noninvasively. However, it is not clear how the dosing would be quantitative or how movement of the vessels with respiration would be accounted for. Experimental work has demonstrated these problems may be addressed.

Of the above noted treatment modalities, only radiofrequency is part of the electromagnetic spectrum. Radiofrequency is penetrating, however its wavelength is significantly longer than visible light, and accordingly, its energy is lower. Shorter wavelengths of the electromagnetic spectrum have higher energy and include ionizing x-ray radiation. Ionizing x-rays are used for medical therapy and diagnostics. Although nerves are considered to be relatively radioresistant, high doses of ionizing x-ray radiation have been used to ablate nerves of the face to treat painful conditions. For example, in the condition known as trigeminal neuralgia, or tic doloureaux, the ganglion the fifth cranial nerve is treated with high doses of radiation in a single session using the Gamma knife instead of microsurgical decompression. Particles therapy such as proton beams can also be used to treat nerves in critical areas. The significant advantage of using x-rays for the therapeutic nerve ablation compared to other nonsurgical treatment modalities, such as radiofrequency, focused ultrasound or cryotherapy, is the x-rays can be used to both image and treat with a precisely determined dosage and volume. Therefore, this single modality can be employed to locate the target and then ablate it with a known volume and dose of energy. This is not possible with other treatment modalities. It is also possible to use ionizing radiation for either imaging or ablation in combination with other imaging modalities and potentially with other ablating modalities. For instance, it is possible to first image the anatomy with magnetic resonance imaging (MRI). The same anatomy can then be imaged via computerized tomography (CT), which uses ionizing x-rays, and the MR and CT images can be fused to better view and localize a target. (Such procedures are commonly used together for radiosurgical treatments to the head.) Because CT images are reconstructed using x-ray radiation attenuation coefficients, the CT numbers in each pixel of an image can be used to plan precise treatments with x-rays. In addition, the imaging capabilities of ionizing x-rays permit images to be captured directly from the treatment beam. Thus, the accuracy of striking the target can be maintained even if the target moves due to respiration or other physiologic processes during therapy.

Filler et al. have described a technique of magnetic resonance (MR) imaging, called MR neurograpy, which uses modified T2 algorithms to specifically image the water in nerves (U.S. Pat. Nos. 5,560,360 and 5,706,813). MR neurographic images can be fused with CT images to better align the relative position of the nerves to be targeted with the surrounding anatomy. In addition, alignment of the internal targets with the external anatomy is achieved by employing surface fiducial markers which are readily visible with MR or CT (x-ray) imaging. Vitamin E capsules secured to a patient's skin are an example of a fiducial system visible with both magnetic resonance and x-ray imaging.

Radiotherapy can be delivered with beams or with seeds, i.e., brachytherapy, employing high-energy, megavoltage, or low-energy, kilovoltage, radiation. In general, low-energy beams are used for us imaging and high-energy beams are used for more penetrating therapy. Existing megavoltage sources, such as those manufactured by Varian Medical Systems, Inc. (Palo Alto, Calif.), Elekta AB (Stockholm, Sweden), Accuray Inc. (Sunnyvale, Calif.) or Siemens Oncology Solutions (Concord, Calif.) can be used to deliver noninvasive nerve ablation in the present invention. It is also possible to use a single x-ray source with adequate kilovoltage energy along with a detector, and software medium and computers to both capture the beam and reconstruct an image and noninvasively treat pathologic lesions in the body. The x-ray source must be capable of moving across the body surface relatively rapidly so as to create an image and minimize x-ray doses to the skin. The beams must be aimed at a lesion so as to concentrate high-dose in the lesion and minimize incidental doses to the normal surrounding tissues. A detector is opposite the source at all times and wide enough to capture the portion of the beam passing through the lesion and normal tissue. The software medium and computers are used to reconstruct the data acquired by the detectors and then plan a safe and effective treatment. Such a system is controlled by the computers and can acquire images prior to treatment as well as during treatment. Thus a known dose of therapeutic radiation is delivered to a known treatment volume noninvasively and capable of accommodating changes in position of the targeted lesion during treatment.

The scanning beam digital x-ray (SBDX) source uses electronic manipulation of a kilovoltage electron beam to rapidly sweep across a tungsten target to create x-rays over a wide surface area. Properly reconfigured, the SBDX source can be used to create a cone-beam or volumetric image and then deliver therapeutic radiation to a selected target. In the present invention, a system using a reconfigured source of ionizing electromagnetic radiation, such as x-rays, is electronically and mechanically moved around a patient to obtain a cone-beam CT image and then plan and treat a pathologic lesion. In the example of treatment of hypertension, there are several approaches for targeting and ablating the contributing neurological pathways involved in the pathology. In this example, the present invention employs ionizing electromagnetic radiation to noninvasively ablate portions of the sympathetic nervous system innervating the kidneys 30 and vasculature. In some embodiments, the paravertebral sympathetic trunk 10 is selectively ablated, either unilaterally or bilaterally with ionizing radiation. The lower thoracic paravertebral trunk, for example T10-T12 and L1 is ablated. In some embodiments, the prevertebral ganglia 22 or collateral ganglia, such as the celiac axis are targeted. In some embodiments, the splanchnic nerves 18, e.g., lesser (comes from T10-T11 and synapses in the aorticorenal ganglion) and least (conies T12 and synapses in the renal plexus) are targeted. In some embodiments, the sympathetic nerves running in the adventitia of the renal arteries are ablated.

Ablation of the sympathetic trunk 10 at the levels of T10-L1 is recited in this invention to control medically refractory hypertension. In some embodiments, ablation of the splanchnic nerves 18 from these spinal levels is performed along with ablation of the prevertebral ganglia 22 innervated by these spinal levels with sympathetic innervation to the viscera, such as the kidney 30. Ablation of the paravertebral ganglia 12 and sympathetic trunk 10 is possible with external radiation as these regions are inaccessible to endovascular techniques. In addition, treating this region can be performed with much lower risk to the normal surrounding tissue. There are fewer vital structures in the region and movement with respiration is more limited and more restricted to this cranio-caudad structure moving in a cranio-caudad direction. On the other hand, including the renal arteries within a beam of external radiation carries a greater danger of damaging the vessels during ablation of the sympathetic nerves running in the outer wall, or adventitia of the artery.

### SUMMARY

In some embodiments, ionizing radiation is delivered to the sympathetic trunk or ganglia to treat hypertension.

In some embodiments, ionizing radiation is delivered to the sympathetic trunk or ganglia from the spinal levels of T10-L1 to treat hypertension.

In some embodiments, ionizing radiation is delivered to the paravertebral ganglia from the spinal levels of T10-L1 to treat hypertension.

In some embodiments, ionizing radiation is delivered to the prevertebral ganglia from the spinal levels of T10-L1 to treat hypertension.

In some embodiments, nonionizing radiation is delivered to the sympathetic trunk or ganglia from the spinal levels of T10-L1 to treat hypertension. In some embodiments, nonionizing radiation is delivered to the paravertebral ganglia from the spinal levels of T10-L1 to treat hypertension.

In some embodiments, nonionizing radiation is delivered to the prevertebral ganglia from the spinal levels of T10-L1 to treat hypertension.

In some embodiments, ionizing radiation is delivered to the sympathetic trunk or ganglia to treat excessive levels of norepinephrine.

In some embodiments, ionizing radiation is delivered to the sympathetic trunk or ganglia to treat excessive sweating, or hyperhidrosis.

In some embodiments, ionizing radiation is delivered to the sympathetic trunk or ganglia to treat vasculitis and Raynaud's syndrome.

In some embodiments, ionizing radiation is delivered to the sympathetic trunk or ganglia to treat painful conditions, such as complex regional pain syndrome, or causalgia, and unremitting cancer pain.

In some embodiments, ionizing radiation is delivered to the sympathetic trunk or ganglia to treat refractory cardiac tachyarrhythmias.

In some embodiments, ionizing radiation is delivered to the sympathetic trunk or ganglia to slow gastrointestinal peristalsis and treat obesity.

In some embodiments, ionizing radiation is delivered to treat peripheral vessels after stenting for peripheral vascular disease to prevent restenosis, or to sites of vascular access, such as AV shunts for renal dialysis, to prevent restenosis.

In some embodiments, ionizing radiation is delivered to treat abnormal nerves.

In some embodiments, ionizing radiation is delivered to treat cancerous masses.

### DESCRIPTION OF FIGURES

FIG. 1 depicts the anatomical relationship of the sympathetic trunk and its branches to be targeted for noninvasive ablation with ionizing radiation to treat hypertension.

### DETAILED DESCRIPTION OF THE INVENTION

The construction and usage of embodiments will become readily apparent from consideration of the following specification as illustrated in the accompanying drawings, in which like reference numerals designate like parts, and wherein:

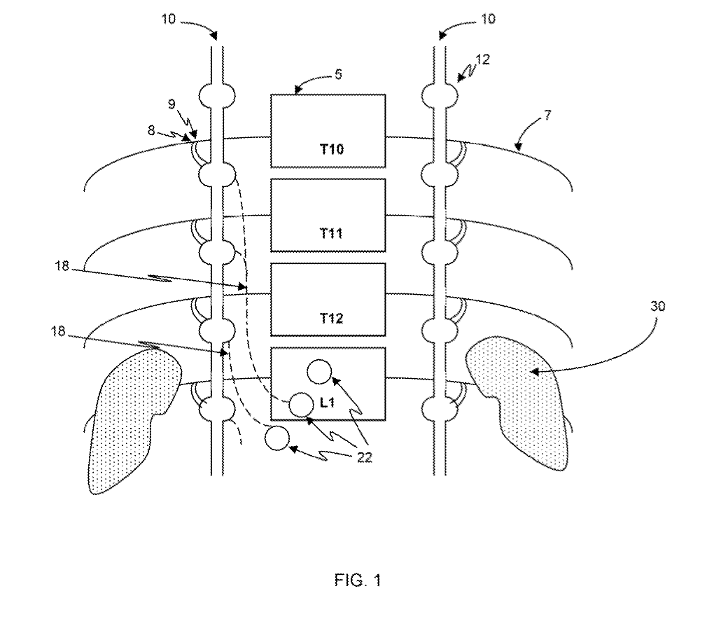

FIG. 1 is a diagram illustrating the anatomy of the sympathetic trunk 10. Potential targets of noninvasive therapeutic ablation including the sympathetic trunk 10 and the paravertebral vertebral ganglia 12, the splanchnic nerves 18, e.g., the lesser and least splanchnic nerves 18, the prevertebral ganglia 22, including the celiac ganglia, superior mesenteric ganglion, aorticorenal ganglion, renal ganglion and inferior mesenteric ganglion. These targets are inaccessible to endovascular techniques and identifying their location within a body is not possible with standard MR imaging protocols or other imaging modalities. To generate and capture clear images of individual nerve structures, MR neurography, with modified T2 algorithms specifically imaging the water in nerves, is first performed to identify and definitively locate the target in this invention. A CT scan is then obtained of the same region and surrounding anatomy. The data is processed so the MR neurography images are then combined, overlaid or fused to the additional CT images via imaging and planning software. Next, a plan is generated directly from a patient's anatomy (i.e., there is no a need for additional internally introduced reference points, cf. U.S. patent application Ser. No. 13/009,784 [0081]) to treat a nerve with ablating beams. Treatment is delivered to a nerve lesion contributing to a disease or symptoms with moving external radiation, e.g., ionizing x-rays conventionally prescribed to a maximum dose of 70 Gy. The standard therapeutic systems used in the methodology of this invention comprise radiation sources and detectors and are associated with imaging and radiation treatment planning software.

Noninvasive nerve ablation is performed according to the plan to treat refractory hypertension, excessive sweating, vasculitis, unremitting painful conditions, refractory tachyarrythmias, obesity, to prevent restenosis in shunts, and mass lesions.

What I claim is:

1. A method comprising:
capturing clear images of individual nerve structures in a body with algorithms specifically imaging water in nerves;
generating additional images of the same region and surrounding anatomy;
processing data to combine the images thereby identifying and locating individual nerves and ganglia within a body;
generating a plan directly from a patient's anatomy to treat a nerve with ablating beams; and
treating a nerve lesion contributing to a disease or symptoms with moving external radiation beams.

2. The method according to claim 1 wherein the nerve structure is selected from the group consisting of the sympathetic trunk and the paravertebral vertebral ganglia from spinal levels of T10-L1, the splanchnic nerves, the lesser and least splanchnic nerves, the prevertebral ganglia, including the celiac ganglia, superior mesenteric ganglion, aorticorenal ganglion, renal ganglion and inferior mesenteric ganglion.

3. The method according to claim I wherein the clear images are obtained with MR neurography and the additional images are obtained with CT scanning.

4. The method according to claim 1 wherein the ablating beam is selected from the group consisting of ionizing x-ray beams having energies ranging from 18 keV to 20 MeV, proton beams, radiofrequency and high-intensity focused ultrasound (HiFU).

5. The method according to claim 1 wherein the disease or symptom is selected from the group consisting of refractory hypertension, excessive sweating, vasculitis, unremitting pain, refractory tachyarrythmias, obesity, restenosis in shunts, and mass lesions.
